(19) United States
(12) Patent Application Publication
(10) Pub. No.: US 2021/0308684 A1
(43) Pub. Date: Oct. 7, 2021

(54) REACTION OR GROWTH MONITORING SYSTEM WITH PRECISION TEMPERATURE CONTROL AND OPERATING METHOD

(71) Applicant: Mango Inc., Cambridge, MA (US)

(72) Inventor: Christopher Davis, Medford, MA (US)

(21) Appl. No.: 17/116,681

(22) Filed: Dec. 9, 2020

Related U.S. Application Data
(60) Provisional application No. 62/945,271, filed on Dec. 9, 2019.

(51) Int. Cl. B01L 7/00; B01L 3/00; G01N 21/03

(52) U.S. Cl. CPC B01L 7/52; B01L 3/50851; B01L 3/50853; G01N 21/0332; B01L 2300/046; G01N 2201/0833; B01L 2300/1822; B01L 2300/1827; B01L 2300/1894; G01N 2201/0826; B01L 2300/0654

(57) ABSTRACT

In a reaction or growth monitoring system, the temperature of a reaction vessel is controlled using heat from a semiconductor sensor placed in direct or thermal contact with the reaction vessel. The heat from the semiconductor sensor is controlled by monitoring the temperature at the reaction vessel and by controlling accordingly, the operation of the sensor and/or by controlling a cooling mechanism in thermal contact with the semiconductor sensor. Additional heat may be provided to the reaction vessel via electromagnetic radiation from an electromagnetic illumination source.

### CROSS-REFERENCE TO RELATED APPLICATIONS

This application claims priority to and benefit of U.S. Provisional Patent Application No. 62/945,271, entitled “A Reaction or Growth Monitoring System with Precision Temperature Control and Operating Method,” filed on Dec. 9, 2019, the entire contents of which are incorporated herein by reference.

### FIELD OF THE INVENTION

This disclosure generally relates to systems for monitoring biological, chemical, and/or biochemical reactions or growth of biologic materials and, in particular, to techniques for precise temperature control of such systems.

### BACKGROUND

Precise control of temperature is critical in the life sciences tests, and biological and chemical reactions, in general. Its critical for the culture of mammalian cells, viruses, prions, microorganisms, as well as for sequence-based reactions such as DNA sequencing, the polymerase chain reaction (PCR), enzymatic reactions, florescent reactions, bioluminescent reactions, molecular probe reactions, binding reactions, and for the precise control of pumps, channels and other components of microfluidic systems.

Precise temperature control can be achieved in two ways—by placing the system o be controlled in a tightly regulated enclosure with much larger thermal mass, essentially overwhelming any temperature fluctuations in the system to be controlled, or by applying precise amounts of heat directly to the item being regulated along with a fast-response temperature sensor in a tight feedback loop.

An example of the first method is the incubator. The incubator includes an insulated box, a heating element, a temperature sensor, and a feedback mechanism to control the power to the heating element so that a precise temperature optimal for growth can be maintained inside the insulated box. Various schemes may also include methods to control humidity, CO2, and other conditions necessary for cell growth. By necessity, any experiment or diagnostic test that requires cells to be grown in a temperature-controlled environment must take place inside of an incubator. This solution is therefore generally suboptimal because an incubator is a large and cumbersome apparatus into which reaction vessels containing cells must be placed and then removed by a person or robotic arm at time intervals for analysis. To avoid the constant removal and replacement of culture dishes, detection instruments, such as a microscope, are sometimes placed inside the incubator to monitor growth changes remotely. The high temperatures, humid environment, and risk of contamination can corrupt organism growth, and corrode instrumentation such as microscope lenses and delicate electronic components common in modern detection systems.

In some procedures, such as DNA sequencing, PCR and other temperature sensitive chemical reactions, not only must these reactions be performed at tightly regulated temperatures, it is necessary to change the temperature rapidly. For these techniques, a reaction vessel such as a PCR tube or multi well plate is placed in contact with a thermally conductive block (usually an alloy of metal). This block is connected to a heating element and/or a cooling element, which is connected to a temperature feedback and control mechanism. This heating block can thus be regulated to a set temperature, or be heated and cooled rapidly to enable or accelerate the reaction inside the reaction vessel. This rapid heating and cooling is usually critical for temperature dependent DNA sequencing, PCR, and other temperature sensitive chemical reactions. Heating and cooling with a conductive block can also be a suboptimal solution because the large thermal mass limits the thermocycling rate, which is limited by the rate of heat dissipation of the thermal block. The block is also large and bulky.

### SUMMARY

In order to minimize size, weigh, bulkiness, and/or complexity of a system used for biological and/or chemical testing, a heat source required to regulate the heating and temperature of a reaction vessel/chamber is either eliminated completely or a smaller heat source that is external to the system may be used. The required heating of the reaction chamber/vessel is achieved, at least in part, from the heat dissipated by the image sensor chip during its operation.

Accordingly, in one aspect, a reaction or growth monitoring system includes a semiconductor sensor and a reaction vessel placed in direct or thermal contact with the semiconductor sensor. The system also includes a cooling mechanism in thermal contact with the semiconductor sensor, and a temperature sensor in thermal contact with the reaction vessel.

The semiconductor sensor may include a digital image sensor having an electronically controllable shutter. The electronically controllable shutter may include several independently controllable shutter groups. Each shutter group may be associated with a respective region of the semiconductor sensor, where ach respective region of the semiconductor sensor is in direct or thermal contact with a respective region of the reaction vessel. It should be understood that direct contact, also referred to as direct physical contact, provides a thermal contact, as well.

The reaction vessel may include a PCR tube, a multi well plate, or a specimen surface. In some embodiments, at least a portion of a top surface of the semiconductor sensor defines at least a portion of a bottom surface of the reaction vessel. The cooling mechanism may include a piezoelectric cooling system or a fan. In some embodiments, the system includes an external, electromagnetic illumination source, configured to emit radiation in a wavelength range from 0.1 up to 1000 μm, for providing additional heat to the reaction vessel. The heat provided by the semiconductor sensor and/or the external heat source is regulated by a processor that obtained a temperature reading of the reaction vessel from the temperature sensor. The processor may also control the operation of the cooling mechanism.

In another aspect, a method is provided for controlling temperature of a reaction vessel. The method includes the steps of heating a reaction vessel from heat emitted by a semiconductor sensor placed in direct or thermal contact with the reaction vessel, and monitoring temperature of the reaction vessel using a temperature sensor. The method also includes controlling the operation of the semiconductor sensor and/or a cooling system in thermal contact with the semiconductor sensor, according to the monitored temperature.

Controlling the operation of the semiconductor sensor may include (i) increasing current passing through the semiconductor sensor for increasing the heat emitted thereby, causing an increase in the temperature of the reaction vessel; or (ii) decreasing the current passing through the semiconductor sensor for decreasing the heat emitted thereby, causing a decrease in the temperature of the reaction vessel. Alternatively, or in addition, controlling the operation of the semiconductor sensor may include: (i) increasing a firing rate of an electronic shutter associated with the semiconductor sensor for increasing the heat emitted thereby, causing an increase in the temperature of the reaction vessel; or (ii) decreasing the firing rate of the electronic shutter associated with the semiconductor sensor for decreasing the heat emitted thereby, causing a decrease in the temperature of the reaction vessel.

In some embodiments, each electronic shutter group in a number of electronic shutter groups is associated with a respective portion of the semiconductor sensor, where the respective portion of the semiconductor sensor is in direct or thermal contact with a respective portion of the reaction vessel. Controlling the operation of the semiconductor sensor may include controlling a firing rate of one or more electronic shutter groups independently of the firing rates of the other shutter groups. As such, the heating of different groups of the reaction vessel that correspond to different image sensor groups may be controlled differently, and different groups of the reaction vessel may be maintained at different selected temperatures.

In some embodiments, controlling the operation of the cooling system includes: (i) turning on the cooling system, (ii) turning off the cooling system, (iii) increasing a rate of cooling of the cooling system, and/or (iv) decreasing the rate of cooling of the cooling system. The method may also include heating the reaction vessel further from an external electromagnetic radiation from an electromagnetic illumination source emitting radiation in a wavelength range from 0.1 up to 1000 μm.

### BRIEF DESCRIPTION OF THE DRAWINGS

The present disclosure will become more apparent in view of the attached drawings and accompanying detailed description. The embodiments depicted therein are provided by way of example, not by way of limitation, wherein like reference numerals/labels generally refer to the same or similar elements. In different drawings, the same or similar elements may be referenced using different reference numerals/labels, however. The drawings are not necessarily to scale, emphasis instead being placed upon illustrating aspects of the invention. In the drawings:

FIG. 1 schematically depicts a reaction/growth monitoring system according to one embodiment;

FIG. 2 depicts an image sensor divided into several regions, according to one embodiment; and

FIGS. 3A and 3B depict two different configurations of a reaction vessel, according to different embodiments.

### DETAILED DESCRIPTION

Semiconductor chips, such as digital image sensors, used in detection technology typically generate excess heat which must dissipate into the environment or be removed by a cooling mechanism such as a piezoelectric cooler. This naturally occurring excess heat can be repurposed to heat the surface of a reaction vessel in direct or near-direct thermal contact with the sensor surface. The reaction vessel may also include the surface of the sensor. This sensor/reaction vessel combination can be coupled to a cooling mechanism such as a piezoelectric cooler and when combined with a temperature feedback mechanism allows for exquisite control of the temperature at the surface of the reaction vessel. Additionally, different regions of the sensor can be heated independently, so as to provide multiple reaction temperatures at different regions within the same reaction vessel.

The types of digital image sensors used in various embodiments may include charge-coupled devices (CCDs), active-pixel sensors (CMOS sensors), fabricated in complementary MOS (CMOS) or N-type MOS (NMOS or Live MOS) technologies, and other charged particle semiconductor sensor. The CCD and CMOS sensors may be based on MOS technology, with MOS capacitors being the building blocks of a CCD, and MOSFET amplifiers being the building blocks of a CMOS sensor. Both types of sensor accomplish the same task of capturing light and converting it into electrical signals.

Each cell of a CCD image sensor is an analog device. When light strikes the chip it is held as a small electrical charge in each photo sensor. The charges in the line of pixels nearest to the (one or more) output amplifiers are amplified and output, then each line of pixels shifts its charges one line closer to the amplifier(s), filling the empty line closest to the amplifiers(s). This process is then repeated until all the lines of pixels have had their charge amplified and output.

A CMOS image sensor (and an image sensor in general) has an amplifier for each pixel compared to the few amplifiers of a CCD. This results in less area for the capture of photons than a CCD, but this problem has been overcome by using microlenses in front of each photodiode, which focus light into the photodiode that would have otherwise hit the amplifier and not been detected. Some CMOS imaging sensors also use back-side illumination to increase the number of photons that hit the photodiode. CMOS sensors can generally be implemented with fewer components, typically use less power, and/or generally provide faster readout than CCD sensors. They are also typically less vulnerable to static electricity discharges.

Another design, a hybrid CCD/CMOS architecture (referred to as “sCMOS”) includes CMOS readout integrated circuits (ROICs) that are bump bonded to a CCD imaging substrate—a technology that was developed for infrared staring arrays and has been adapted to silicon-based detector technology. Another approach is to utilize the very fine dimensions available in modern CMOS technology to implement a CCD like structure entirely in CMOS technology: such structures can be achieved by separating individual poly-silicon gates by a very small gap. The hybrid sensors can harness the benefits of both CCD and CMOS imagers.

Measuring the temperature at the reaction vessel surface using a thermistor or other fast response temperature sensing device provides input into a control mechanism which can activate and/or control the sensor to generate heat and/or activate and/or control the piezoelectric cooler to cool the system. Because temperature is monitored at the reaction surface, a precise temperature can be controlled by turning on or by controlling the operation of the sensor, e.g., by passing more or less current to the sensor or, in the case of the CMOS sensor or CCD sensor, by controlling the firing rate/frequency of an electronic shutter associated with the sensor, and/or by turning on/off or by controlling the cooling system.

Conventionally, physical separation of incubation and detection systems was required because sensitive detection technologies such as lenses or other pieces of instrumentation necessitated a substantial distance between the semiconductor chip and the reaction vessel. As such, the heat generated by a semiconductor chip could not be exploited to heat the reaction vessel by providing a direct or a thermal contact between the semiconductor chip and the reaction vessel for heating and/or cooling the vessel.

With the recent advances in lens-free imaging technology, a reaction vessel such as a cell culture vessel can be placed in direct contact with (or in close proximity to) a CMOS sensor responsible for imaging cells. In some cases, the sensor surface itself can form a part of the reaction vessel. In some embodiments, the sensor and thus the vessel is cooled using cooling mechanisms such as a piezoelectric cooling system. Temperature at the reaction surface may be monitored by a thermistor or other temperature sensing device and this information may be provided to a feedback mechanism which controls heating and cooling of the sensor to maintain a precise temperature at the reaction surface.

Integrating heat regulation via thermal conduction and, optionally, by thermal radiation in addition, for incubation and/or thermocycling into a detection instrument avoids the need for placement and removal of cell culture dishes into the incubator. Other advantages include reducing the size of the combined reaction vessel and the sensor instrument. By combining incubation with the sensing instrument, temperature can be controlled with greater precision and the number of parts can be reduced in the combined system, thus reducing the failure points, and also reducing the footprint of the incubation/detection system.

In traditional incubators or thermocyclers all reaction vessels are generally maintained at a single temperature. Also, using the conventional techniques, the temperature at different regions within a single reaction vessel cannot be adjusted to different values. All the subregions generally can be maintained at the same temperature only. According to some embodiments described herein, however, a system having multiple reaction vessels and sensors can be provided where each subunit having a reaction vessel or a portion thereof and a corresponding sensor or a portion thereof, and where the temperature of each subunit can be controlled individually. Additionally, precise control of temperature at subregions of a reaction vessel can be performed by controlling the operation of a corresponding subregion of the sensor.

Various embodiments described herein avoid the use of an external incubator or separate heating block or element. The reaction vessel is placed in thermal contact with the semiconductor sensor chip, which is in thermal contact with a cooling element. The heat from the sensor chip itself can be used beneficially for heating the reaction vessel. Instead of providing a distinct reaction vessel, in some embodiments, the reaction vessel is integrated with the sensor, where the sensor surface itself forms a surface of the reaction vessel. The sensor surface may include a pixel array surface, the color filter array surface, the micro-lens array surface, a light pipe, a surface coating, or a cover glass.

In various embodiments, the temperature at the reaction surface is controlled by exploiting heat already emitted by the detection sensor. The rate of heat generation can be modulated by increasing or decreasing the current passing through the sensor. If needed, additional heat may be generated by thermal radiation emitted from an electromagnetic illumination source, e.g., a source emitting radiation in the wavelength range from 0.1 up to 1000 μm. On a CMOS or CCD sensor (an image sensor, in general) heat generating current can delivered to a subset of pixels, allowing precise control of temperature in subregions of the imaging sensor. The temperature can be decreased by activating an active or passive cooling mechanism in direct or thermal contact with the sensor such as a piezoelectric cooler. In some embodiments, precise control of the temperature at a particular region of the reaction vessel can be achieved by passing current to a subset of elements in a sensor. For example one or more photodiodes in a particular region of a CMOS or CCD sensor can allow for temperature of the portion of the reaction vessel that is directly over or closest to the particular region of the sensor. Thus, different portions of the reaction vessel can be simultaneously maintained at different temperatures by controlling the operation of different regions of the sensor.

In microfluidics systems this technique allows for the precise control of subcomponents of the microfluidic system. This includes, but is not limited to one or more of the following: micropumps, micromixers, valves, separators and concentrators. Pumps, valves, separators, and concentrators may all be controlled by thermal activation. This includes precise control of reaction rates and flow rates.

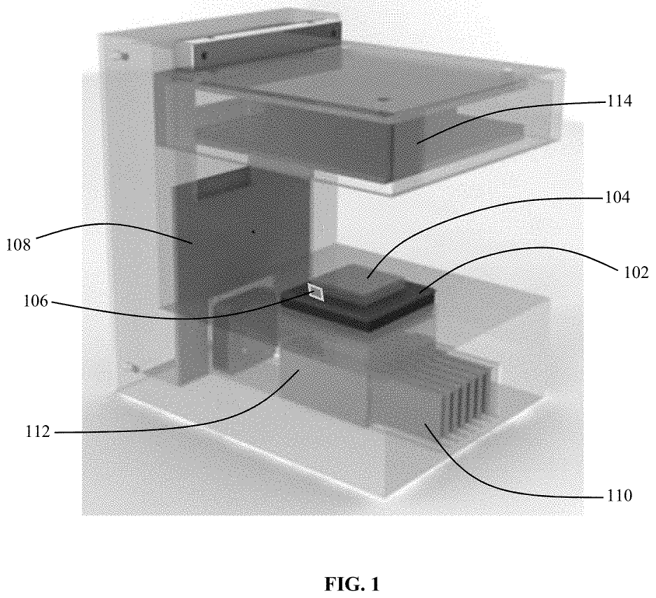

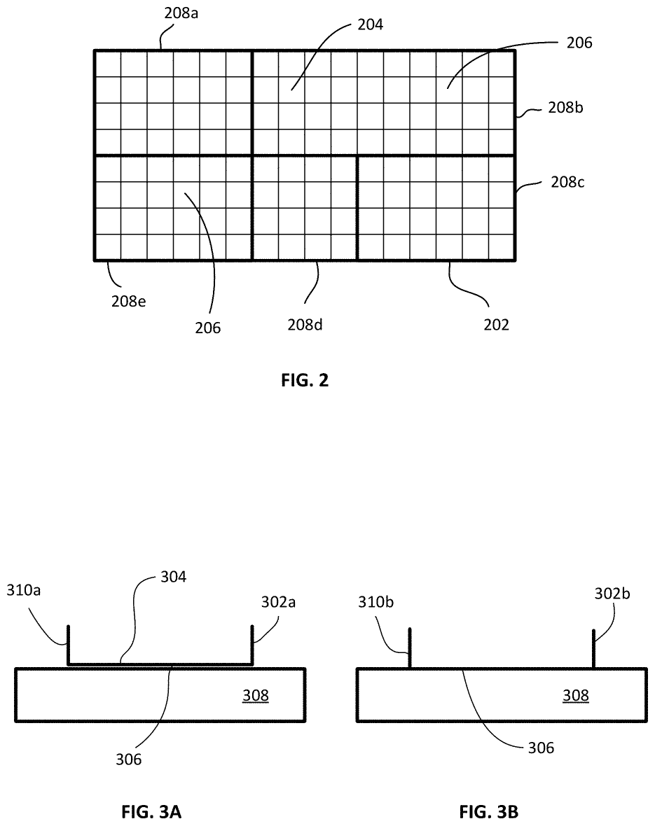

With reference to FIG. 1, in a reaction or growth monitoring system 100, electrical current passes through an image sensor 102 which generates heat. To heat a region of an image sensor such as a CCD or CMOS sensor, photoelectric conversion and charge accumulation are activated for all the pixels of the image sensor or in one or more subsets of pixels, described below with reference to FIG. 2. The subsets of pixels can be defined in software or firmware which, can also determine which subset of pixels is to be activated when an electronic shutter associated with the sensor is triggered. The shutter can be triggered all at once, for all pixels of the image sensor, or different sections of the shutter can be triggered at different times and/or at different rates.

The heat generated by the sensor passes to the reaction vessel 104 via thermal conduction and/or thermal convection, and the reaction surface is heated, as described below with reference to FIG. 3. The temperature of the reaction surface is monitored by a temperature monitoring device 106 (e.g., a temperature sensor) on or near the surface of the reaction vessel 104. The monitoring device/temperature sensor 106 is placed in thermal contact with the reaction vessel 104 (e.g., with the bottom surface of the reaction vessel) and/or with the image sensor 102 (e.g., the top surface of the image sensor). The thermal contact can be provided via a direct physical contact and/or via an intervening thermally conductive material, such as a metallic element (a block, wire, etc.) or a thermally conductive paste.

The temperature monitoring device/sensor 106 is a different type of sensor from the image sensor 102. The sensor 106 does not perform image sensing as the image sensor 102 does and the image sensor 102 typically does not perform temperature sensing. More than one temperature monitoring devices/sensor 106 may be used to measure temperature at different regions of the image sensor 102 and/or the corresponding regions of the reaction vessel 104.

The temperature value sensed by the monitoring device/sensor 106 is passed to a control board 108 with a processor programmed to maintain at the reaction surface (or a selected region thereof) a predetermined temperature. The processor on the board 108 controls the temperature of the reaction vessel by increasing current passing through the sensor to heat the reaction vessel. To cool the reaction vessel, the control board can reduce the current and, additionally, may activate a cooling mechanism 110 which cools the reaction vessel 104 by cooling the image sensor 102. The cooling mechanism, in general, may include a solid-state thermoelectric cooling system, a refrigerant based cooling system, a piezoelectric cooling system or a fan.

The cooling mechanism 110 may be placed in physical contact with a thermally conductive element 112 (e.g., a metallic block), which is in physical contact with the image sensor 102. In some embodiments, the cooling mechanism is placed in direct physical contact with the image sensor 102. In both cases, the cooling mechanism is in thermal contact with the image sensor 102, and can thus cool the image sensor by dissipating heat generated by the image sensor 102.

In some embodiments, the heat generated by the image sensor 102 (also referred to as the semiconductor sensor) is sufficient to raise the temperature of the reaction vessel 104 to the desired level. In other embodiments, another heating element 114 may be used together with the semiconductor sensor chip 102. In some embodiments, additional heat may be provided by electromagnetic radiation from an illumination source or other external source of electromagnetic radiation. In some embodiments that use CMOS sensors, the frame rate of the electronic shutter is modulated. Sensors other than CMOS or CCD sensors may be controlled by controlling the clock rate and/or supply voltage.

In some embodiments, the entire surface of the image sensor is heated above the ambient temperature by exploiting the firing rate of the electronic shutter. When the instrument is at room temperature, to maintain a temperature around 37° C. the electronic shutter of the CMOS sensor is fired at a rate of 64 times every 3 minutes. The temperature can be maintained at 50° C. by increasing the rate of triggering the shutter, while still collecting submicron resolution images with acceptable levels of noise. In some embodiments, the image sensor and reaction surface temperature are lowered by activating a fan that circulates ambient air around a heatsink which is coupled to a camera board (e.g., the control board 108) which is coupled via thermal paste to the digital image sensor 102.

In some embodiments, the CMOS sensor 102 is decoupled from the camera board and a socket with Pogo pins is the interface between the camera board and the wire bonding of the CMOS sensor. This socket is aluminum and can act as a temperature stabilizing thermal block. Some embodiments employ an infrared thermometer, that need not be in contact with the image sensor 104 and/or the reaction vessel 104, but can nevertheless measure the temperature at the surface of the reaction vessel, and can thus replace the temperature sensor 106. One or more infrared sensors can be used in addition to the temperature sensor 106, and can measure temperatures at different regions of the image sensor 102 and/or the corresponding regions of the reaction vessel 104.

With reference to FIG. 2, a semiconductor image sensor 202 has a sensing surface 204, which includes sensing pixels 206. The surface 204 is divided into regions 208a-208e. It should be understood that the number, sizes, and shapes of the regions depicted in FIG. 2 are illustrative only and that a sensor surface, in general, can have any number of regions, and such regions can have any shape, including non-rectangular shapes, such as circular or ovular shapes. The regions of the sensor surface can define the corresponding regions of the reaction vessel disposed over and in thermal contact with the sensor surface. In some cases, the entire semiconductor image sensor is not divided into regions, which can be understood has the image sensor having a single region. Correspondingly, the reaction vessel may also have no distinct regions or, equivalently, may have only one region.

The operation of the semiconductor image sensor 202 can be controlled by increasing or decreasing the current passing through the entire semiconductor sensor 202. Alternatively, the current passing through each region of the image sensor 202 may be controlled independently of the other regions. Increasing the current passing through an image sensor (or a region thereof) generally increases the heat emitted by the image sensor (or the region thereof), causing an increase in the temperature of the reaction vessel (or in the corresponding region of the reaction vessel). Decreasing the current passing through an image sensor (or a region thereof) generally decreases the heat emitted by the image sensor (or the region thereof), causing a decrease in the temperature of the reaction vessel (or in the corresponding region of the reaction vessel). In some embodiments, the current supplied to different regions of the image sensor 202 is controlled by the processor on a control board independently of the current supplied to the other regions.

Electronically controllable shutters that respectively correspond to the regions 208a-208e may be provided with the image sensor 202. The firing rate of each shutter may be electronically controllable independent of the firing rates of the other shutter. Increasing a firing rate of an electronic shutter associated with a particular region of the semiconductor image sensor 202 can increase the heat emitted from that region, causing an increase in the temperature of the corresponding region of reaction vessel. In contrast, decreasing the firing rate of the electronic shutter associated with a particular region of the semiconductor image sensor 202 can decrease the heat emitted from that region, causing a decrease in the temperature of the corresponding region of the reaction vessel. In some cases, only a single electronically controllable shutter may be provided with the image sensor 202, but the currents supplied to the different regions may be controlled differently. In some cases, the control of the current is not region-specific but the firing of the respective shutters associated with the different regions of the image sensor is controlled differently. In some cases, both the currents supplied to different regions and the firing of the respective shutters are controlled individually for the different regions.

The configuration described above facilitates different types of biologic and/or chemical reactions in different regions where such reactions/growth require different regions of the vessel to be maintained at different temperatures. Specifically, not only the temperature of the entire vessel but also of different regions of the vessel can be rapidly cycled between multiple temperatures, using the configurations described above.

With reference to FIG. 3A, a reaction vessel 302a having a distinct bottom surface 304 is affixed to the top surface 306 of an image sensor 308. The reaction vessel 302a also has walls 310a. With reference to FIG. 3B, a reaction vessel 302b does not have a distinct bottom surface and is defined only by the walls 310b affixed to the top surface 306 of the image sensor 308. In this case, the top surface 306 of the image sensor 308 defines the bottom surface of the reaction vessel 302b. In both cases, the reaction vessel is in direct physical contact and, thus, in thermal contact, with the image sensor 308. In some cases, the reaction vessel 302a may be placed over a transparent thermally conductive material, such a thermally conductive paste or glue, which is in physical contact with the upper surface 306 of the image sensor 308. Thus, in these cases also, the reaction vessel 302a is in thermal contact with the image sensor 308.

If the top surface 306 of the image sensor 308 is divided into several regions (as described with reference to FIG. 2), the reaction vessels 302a, 302b may also include corresponding reaction regions. in particular, the bottom surface of 304 of the reaction vessel 302a may be considered to have similar regions corresponding to the regions of the top surface 306 of the image sensor 308. Since the reaction vessel 302b does not have a distinct bottom surface, the different regions of the top surface 306 of the image sensor 308 may define different regions of the reaction vessel 302b.

A computing system, control board, or processor used to implement various embodiments may include general-purpose computers, vector-based processors, graphics processing units (GPUs), network appliances, mobile devices, or other electronic systems capable of receiving network data and performing computations. A computing system in general includes one or more processors, one or more memory modules, one or more storage devices, and one or more input/output devices that may be interconnected, for example, using a system bus. The processors are capable of processing instructions stored in a memory module and/or a storage device for execution thereof. The processor can be a single-threaded or a multi-threaded processor. The memory modules may include volatile and/or non-volatile memory units.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. Various embodiments and functional operations and processes described herein may be implemented in other types of digital electronic circuitry, in tangibly-embodied computer software or firmware, in computer hardware, including the structures disclosed in this specification and their structural equivalents, or in combinations of one or more of them.

A control board/processor may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit). Computers/processor suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer/processor need not have such devices. Moreover, a computer/processor can be embedded in another device, e.g., a mobile telephone, a laptop, a desktop, a tablet, etc.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

What is claimed is:

1. A reaction or growth monitoring system, comprising:
a semiconductor sensor;
a reaction vessel placed in direct or thermal contact with the semiconductor sensor;
a cooling mechanism in thermal contact with the semiconductor sensor; and
a temperature sensor in thermal contact with the reaction vessel.

2. The system of claim 1, wherein the semiconductor sensor comprises a digital image sensor having an electronically controllable shutter.

3. The system of claim 2, wherein:
the electronically controllable shutter comprises a plurality of independently controllable shutter groups;
each shutter group is associated with a respective region of the semiconductor sensor; and
each respective region of the semiconductor sensor is in direct or thermal contact with a respective region of the reaction vessel.

4. The system of claim 1, wherein the reaction vessel comprises a PCR tube, a multi well plate, or a specimen surface.

5. The system of claim 1, wherein at least a portion of a top surface of the semiconductor sensor defines at least a portion of a bottom surface of the reaction vessel.

6. The system of claim 1, wherein the cooling mechanism comprises a piezoelectric cooling system or a fan.

7. The system of claim 1, further comprising:
an electromagnetic illumination source, emitting radiation in a wavelength range from 0.1 up to 1000 μm, for providing additional heat to the reaction vessel.

8. A method for controlling temperature of a reaction vessel, the method comprising the steps of:
heating a reaction vessel from heat emitted by a semiconductor sensor placed in direct or thermal contact with the reaction vessel;
monitoring temperature of the reaction vessel using a temperature sensor; and
controlling operation of the semiconductor sensor or a cooling system in thermal contact with the semiconductor sensor according to the monitored temperature.

9. The method of claim 8, wherein controlling the operation of the semiconductor sensor comprises one of:
(i) increasing current passing through the semiconductor sensor for increasing the heat emitted thereby, causing an increase in the temperature of the reaction vessel; or
(ii) decreasing the current passing through the semiconductor sensor for decreasing the heat emitted thereby, causing a decrease in the temperature of the reaction vessel.

10. The method of claim 8, wherein controlling the operation of the semiconductor sensor comprises one of:
(i) increasing a firing rate of an electronic shutter associated with the semiconductor sensor for increasing the heat emitted thereby, causing an increase in the temperature of the reaction vessel; or
(ii) decreasing the firing rate of the electronic shutter associated with the semiconductor sensor for decreasing the heat emitted thereby, causing a decrease in the temperature of the reaction vessel.

11. The method of claim 8, wherein:
each electronic shutter group in a plurality of electronic shutter groups is associated with a respective portion of the semiconductor sensor, the respective portion of the semiconductor sensor being in direct or thermal contact with a respective portion of the reaction vessel; and
controlling the operation of the semiconductor sensor comprises controlling a firing rate of a first electronic shutter group independently of firing rates of the other shutter groups.

12. The method of claim 8, wherein controlling the operation of the cooling system comprises one of: (i) turning on the cooling system, (ii) turning off the cooling system, (iii) increasing a rate of cooling of the cooling system, or (iv) decreasing the rate of cooling of the cooling system.

13. The method of claim 8, further comprising:
heating the reaction vessel further from electromagnetic radiation from an electromagnetic illumination source emitting radiation in a wavelength range from 0.1 up to 1000 μm.
